(19) United States
(12) Patent Application Publication
(10) Pub. No.: US 2026/0295176 A1
(43) Pub. Date: Oct. 1, 2026

(54) PATIENT-SPECIFIC NEEDLE GUIDE

(71) Applicant: BOARD OF REGENTS, THE UNIVERSITY OF TEXAS SYSTEM, Austin, TX (US)

(72) Inventors: Sujaya H. RAO, Houston, TX (US); Karthik Kumar TAPPA, Houston, TX (US); Carlos ROLDAN, Houston, TX (US)

(21) Appl. No.: 19/633,411

(22) Filed: Mar. 30, 2026

Related U.S. Application Data
(60) Provisional application No. 63/780,721, filed on Mar. 31, 2025.

(51) Int. Cl. A61M 5/32; A61M 5/46

(52) U.S. Cl. CPC A61M 5/3287; A61M 5/46

(57) ABSTRACT

The present disclosure relates to systems and methods for patient-specific needle guidance. For example, a method of preparing a patient-specific needle guide may include obtaining three-dimensional data from imaging a patient's anatomy. The method may also include identifying, in the three-dimensional data, a target treatment location and a three-dimensional surface geometry. The method can further include using the identified target treatment location and the identified three-dimensional surface geometry to define a patient-specific needle guide geometry. The patient-specific needle guide geometry may include at least one guide surface that is contoured based on the three-dimensional surface geometry and a needle receiving guide that is positioned and oriented relative to the at least one guide surface based on the target treatment location. Additionally, the method may include using the defined patient-matched needle guide geometry to manufacture the patient-specific needle guide.

### CROSS REFERENCE TO RELATED APPLICATIONS

This application claims priority to and the benefit of the filing date of U.S. provisional patent application Ser. No. 63/780,721, filed Mar. 31, 2025 for a Patient-Specific Needle Guide, the entire contents of which are hereby incorporated by this reference.

### FIELD OF THE DISCLOSURE

Embodiments of the present disclosure relate generally to a patient-specific needle guide.

### BACKGROUND

Injection of a local anesthetic, other medication, or other injectable into a joint, nerve, or other target anatomy is a commonly performed procedure. For instance, injection of a local anesthetic or other medication into a joint or nerve is often used as an effective and reliable treatment for pain management. When administering the injection, a needle may be guided to a deep anatomical target associated with a target joint or nerve using an imaging modality (e.g., ultrasound, x-ray, fluoroscopy, computed tomography, or magnetic resonance imaging). In current practice, use of such an imaging modality at the time of injection may be necessary to ensure precise and accurate needle placement during the injection. For example, in many current practices, if an imaging modality is not used during injection, the needle may penetrate to an inaccurate depth or at a nonoptimal angle. There are risks inherent to a lack of precise and accurate needle placement during an injection of a target joint or nerve. For example, the lack of precision may cause the needle to damage tissue or other anatomical structures that are not part of the target joint or nerve, which can cause bleeding, infection, worsening pain, or additional injuries to the tissue or other structures. However, it is not always possible or efficient to utilize an imaging modality every time such an injection is required. Systems and methods for facilitating controlled and precise needle placement without in situ use of an imaging modality are desirable.

### BRIEF SUMMARY

This disclosure relates to systems and methods for patient-specific needle guidance. This summary is not an exhaustive overview. This summary is not intended to identify key, critical, and/or essential elements of the methods and systems disclosed herein or to fully delineate the scope of this disclosure. This simplified summary is a prelude to the more detailed description presented in the Detailed Description below.

In one example, a method of preparing a patient-specific needle guide is provided that includes obtaining three-dimensional data from imaging a patient's anatomy; identifying in the three-dimensional data a target treatment location associated with a deep anatomical structure of the patient's anatomy; identifying in the three-dimensional data a three-dimensional surface geometry associated with an external surface of the patient's anatomy; using the identified target treatment location and the identified three-dimensional surface geometry to define a patient-specific needle guide geometry comprising (i) at least one guide surface that is contoured based on the three-dimensional surface geometry; and (ii) a needle receiving guide that is positioned and oriented relative to the at least one guide surface based on the target treatment location; and using the defined patient-matched needle guide geometry to manufacture the patient-specific needle guide.

In some examples, the method further includes identifying in the three-dimensional data a needle insertion location relative to the three-dimensional surface geometry. The needle insertion location may also be used to define the patient-specific needle guide geometry.

In some examples, the method includes identifying in the three-dimensional data a needle insertion location. In such examples, the needle insertion location may be identified such that a needle trajectory extending from the needle insertion location to the deep target treatment location does not pass through the second location.

In some examples, the defined patient-specific needle guide geometry further comprises a needle depth stop that is defined based on the target treatment location and the needle insertion location.

In some examples, the method includes using the manufactured patient-specific needle guide to guide an injection procedure on the patient. In such examples, the method can further include positioning the patient-specific needle guide such that the guide surface contacts the external surface of the patient and inserting a needle through the needle receiving guide such that a distal tip of the needle penetrates to the deep anatomical structure associated with the target treatment location.

In some examples, the method may further include using the needle to deliver a pharmacological agent to facilitate a nerve block or to deliver a neurolytic to the target treatment location.

In some examples, the target treatment location includes a nerve or a bone joint.

In some examples, the method may further include generating a 3D model representative of the 3D data obtained from imaging the patient's anatomy.

In some examples, the target treatment location and the three-dimensional surface geometry are identified using the 3D model.

In some examples, using the defined patient-matched needle guide geometry to manufacture the patient-specific needle guide includes using a three-dimensional (3D) printer to 3D print the patient-specific needle guide.

In some examples, obtaining the three-dimensional data from imaging the patient's anatomy includes obtaining the three-dimensional data from a plurality of MRI images or a plurality of CT images.

In some examples, the three-dimensional data comprises an imaging measurement and a position in three-dimensional space.

In another example, a patient-specific needle guide incudes at least one guide surface that is contoured to correspond to an external surface of a patient, wherein the external surface is associated with a target treatment location in that the target treatment location is reachable by a needle from the external surface; and a needle receiving guide that is positioned and oriented relative to the at least one guide surface based on the target treatment location.

In some examples, the at least one needle receiving guide is further positioned relative to a needle insertion location on the external surface.

In some examples, the patient-specific needle guide further includes a needle depth stop that is configured to prevent insertion of a needle past a maximum depth. The needle depth stop may be positioned on the needle receiving guide based on the target treatment location and the needle insertion location.

In some examples, a length of the needle receiving guide is based on the target treatment location and the needle insertion location.

In some examples, a diameter of the needle receiving guide is smaller than a diameter of a syringe associated with the needle.

In some examples, the patient-specific need guide further comprises a second needle receiving guide that is positioned and oriented relative to the at least one guide surface based on a second target treatment location.

### BRIEF DESCRIPTION OF THE DRAWINGS

FIG. 1 is a schematic example of three-dimensional (3D) data obtained by imaging a patient's anatomy according to some aspects of the present disclosure.

FIG. 2 is the schematic example of the 3D data obtained by imaging the patient's anatomy with a treatment location identified.

FIG. 3 is the schematic example of the 3D data obtained by imaging a patient's anatomy being used to define a patient-specific needle geometry.

FIG. 4 is a schematic example of a patient-specific needle guide according to some aspects of the present disclosure.

FIG. 5 is another schematic example of a patient-specific needle guide according to some aspects of the present disclosure.

FIG. 6 is a block diagram of an example of a system for preparing a patient-specific needle guide according to some aspects of the present disclosure.

FIG. 7 is a flowchart of an example of a method for preparing a patient-specific needle guide according to some aspects of the present disclosure.

FIG. 8 is a block diagram of an example of a computing device for preparing a patient-specific needle guide according to some aspects of the present disclosure.

### DETAILED DESCRIPTION

This disclosure relates to systems and methods for patient-specific needle guidance. For example, embodiments of the present disclosure provide a method of preparing a patient-specific needle guide. The patient-specific needle guide may be used in an injection procedure on a patient (e.g., an injection of a joint or nerve for pain treatment). The patient-specific needle guide may be customized to an anatomy of the patient to facilitate safe, precise, and efficient injection procedures.

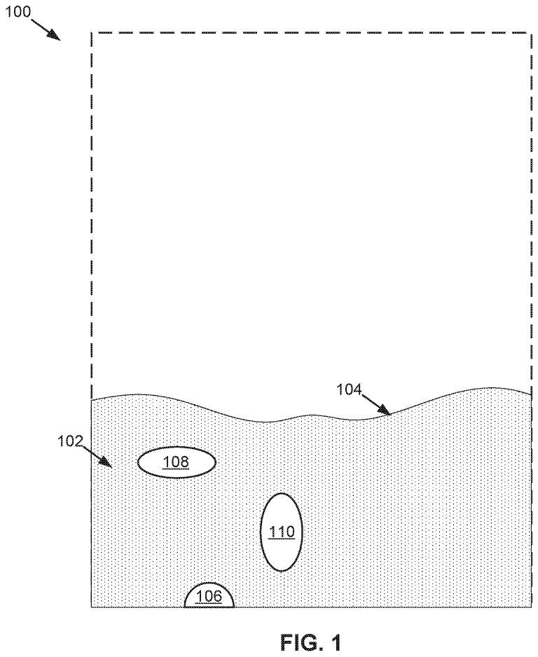

FIG. 1 shows an example of a schematic representation of three-dimensional (3D) data obtained by imaging a patient's anatomy according to some aspects of the present disclosure. Although FIG. 1 is two-dimensional, FIG. 1 is being used to schematically represent a 3D model 100. The 3D model 100 may be a visual representation of the 3D data obtained by the imaging of the patient's anatomy. The patient's anatomy may be imaged using a medical imaging modality such as x-ray imaging, computed tomography (CT) imaging, magnetic resonance imaging (MRI), ultrasound, or the like. The patient's anatomy may include an external surface (e.g., a skin surface of the patient) and a treatment site (e.g., a deeper structure to be treated for pain management purposes via an injection). The 3D model 100 therefore shows an external surface 104 of the patient's anatomy and a corresponding deep anatomical area 102 that includes deep anatomical structures 106, 108, 110. The deep anatomical structures 106, 108, 110 may include any anatomical structure under the skin of a patient. For example, the deep anatomical structures 106, 108, 110 be part of a joint, nerve, muscle, vein, or another anatomical structure.

In some examples, the medical imaging modality may be used to obtain a set of slices (e.g., two-dimensional cross-sectional images) of the patient's anatomy. The imaging modality (e.g., CT or MRI) may obtain each slice of the patient's anatomy at substantially regular intervals and in an anatomical plane (e.g., in an axial plane, a coronal plane, or a sagittal plane) with respect to the body of the patient. Each slice may be labeled with metadata indicative of an orientation of the slice based on the anatomical place, a thickness of the slice, a spacing distance between the slice and a subsequent and/or proceeding slices, or other spatial information.

The set of slices may then be used to ascertain the 3D data schematically represented in FIG. 1. For example, the spatial information (e.g., the slice thickness, the spacing distance between adjacent slices, and the slice orientation) may be used in combination with pixel data and with manual or automated segmentation or other image processing to obtain the 3D data and/or construct the 3D model 100. The pixel data may include an imaging measurement associated with each pixel of each slice. The imaging measurement may be obtained via the medical imaging modality. For example, the imaging measurement may be a tissue density measurement in CT or signal intensity measurement in MRI. The combination of pixel data and spatial information may be represented as voxels (e.g., 3D pixels) that each represent a single unit of volume (e.g., a cube or other 3D unit of tissue) within a 3D space (e.g., the patient's anatomy). Thus, the 3D data may be represented as voxels.

Generating the 3D model 100 may include preprocessing each slice (e.g., each two-dimensional cross-sectional image) by filtering to remove artifacts in each slice, enhancing contrast to improve visibility of one or more deep anatomical structures in at least some of the slices, or by performing other preprocessing techniques. Generating the 3D model 100 may further include performing segmentation on the slices to identify regions of interest (e.g., the external surface 104 and deep anatomical structures 106, 108, 110). To do so, the slices may be input into a machine learning model (e.g., a neural network, a decision tree, a support vector machine, or the like) trained to predict which pixels in each slice are associated with a region of interest. Additionally, or alternatively, the segmentation can include executing an algorithm that identifies a region of interest based on an imaging measurement (e.g., intensity) of corresponding pixels in each slice being within in a measurement range (e.g., an intensity range). Additionally, or alternatively the segmentation or other processing may involve manual or at least semi-automated techniques. Other techniques for identifying the regions of interest may also be used within the scope of the present disclosure.

After the preprocessing and/or segmentation of the slices, the 3D model 100 can be constructed by compiling the preprocessed and/or segmented set of slices into a 3D volume. 3D model construction tools (e.g., 3D slicer, OsiriX, Amira, or the like) and/or 3D model construction algorithms (e.g., marching cubes or surface rendering) may be employed to construct the 3D model 100 from the set of slices and the spatial information (e.g., the slice thickness, spacing distance between adjacent slices, and slice orientation). The 3D model 100 may also be refined to enhance accuracy and improve usability. For example, the 3D model 100 may be refined by implementing one or more smoothing algorithms (e.g., Laplacian smoothing, bilateral filtering, etc.), mesh simplification, artifact removal, texture and/or color mapping, model scaling, other refinement techniques, or a combination thereof.

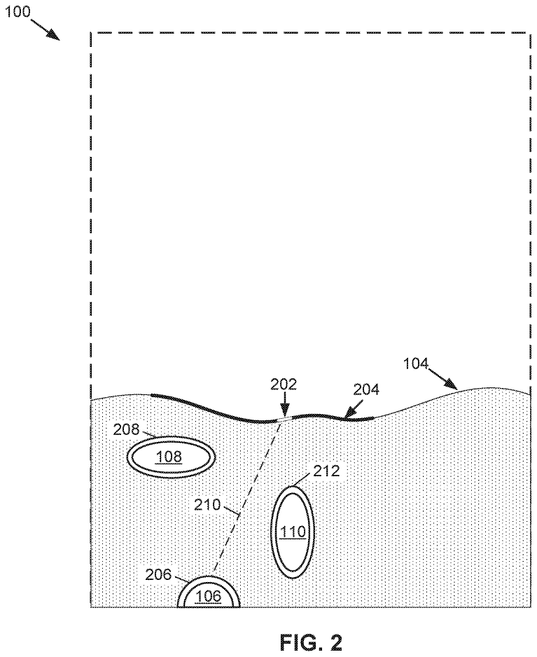

FIG. 2 is the schematic representation of the 3D data with a target treatment location 206 identified. The identification of the target treatment location 206 using the 3D data may involve locating and identifying the segmented structure 106 in the 3D model 100 using automated, semi-automated, or manual input. In one example, the deep anatomical structure 106 is a nerve that is the target injection site for an injection procedure (e.g., a nerve block injection). Thus, the target treatment location 206 can indicate the location of the nerve to facilitate the injection procedure.

A second location 208 and a third location 212 may also be identified using the 3D data. For example, the use of segmentation during the construction of the 3D model 100 may result in the additional identification of the deep anatomical structures 108, 110 in FIGS. 1 and 2. Those deep anatomical structures 108, 110 may be subsequently designated (again, using automated, semi-automated, or manual techniques) in the 3D model. In contrast to the target treatment location 206, the second location 208 and the third location 212 may indicate sites to avoid during the injection procedure. For example, the deep anatomical structure 108 and the deep anatomical structure 110 may be a blood vessel, an area along the spinal cord, an artery, or another region to avoid during the injection procedure to prevent unintended injury or damage to other structures.

A 3D surface geometry 204 is also identified in the 3D data. The 3D surface geometry 204 may include a three-dimensional shape that corresponds to the external surface 104 (e.g., a skin surface) of the patient. Identifying the 3D surface geometry 204 from the 3D data, may include identifying the 3D surface geometry 204 in the 3D model 100 that is representative of the 3D data. For example, the external surface 104 of the 3D model 100 is shown in FIGS. 1 and 2. A section of the external surface 104 may then be selected as being closest to the intended target treatment location 206 or as including a needle insertion location 202. The 3D surface geometry 204 may then be defined based on the selected section of the external surface 104 provided in the 3D model 100.

Additionally, the needle insertion location 202 relative to the 3D surface geometry 204 can be identified in the 3D data. The needle insertion location 202 may correspond to a relatively small surface area of the skin of the patient through which a needle is to be inserted. The needle insertion location 202 may be identified by identifying a relatively small surface area on the external surface 104 from which a needle trajectory 210 can extend to the target treatment location 206 without passing through the second location 208 or the third location 212. It may be desirable to select and identify the needle insertion location 202 such that the needle trajectory 210 does not pass through the second location 208 or the third location 212 to prevent damage or other undesirable effects of contact of a needle with either of the deep anatomical structures 108, 110.

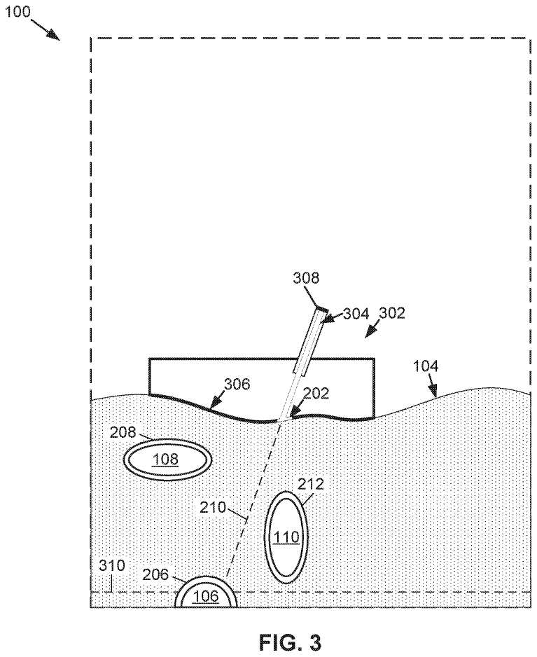

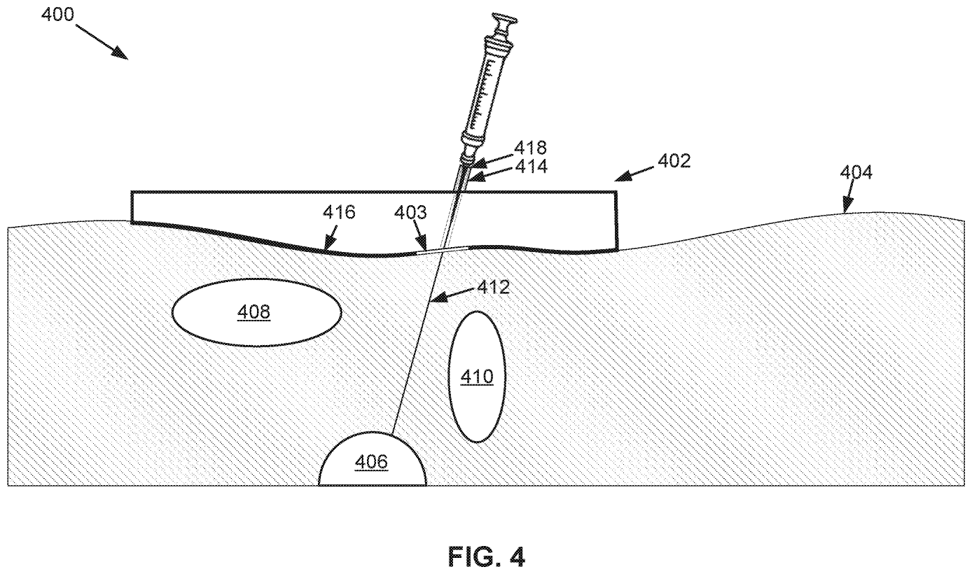

FIG. 3 is the schematic representation of the 3D data being used to define a patient-specific needle guide geometry 302. The patient-specific needle guide geometry 302 includes a guide surface 306. The guide surface 306 may be a substantially thin and plate-like structure that is contoured to the external surface 104 of the patient. The 3D surface geometry 204 may be used to generate the guide surface 306 that is contoured to the external surface 104. The external surface 104 to which the guide surface 306 is contoured may be associated with the target treatment location 206 in that the target treatment location 206 can be reached by a needle from the external surface 104. An example of the target treatment location being reached by a needle from the skin surface is shown and described below with respect to FIG. 4.

The patient-specific needle guide geometry 302 further includes a needle receiving guide 304. The needle receiving guide 304 may be a structure that defines an opening in the guide surface 306 through which a needle can be inserted. The needle receiving guide 304 may further protrude from the guide surface 306, as shown in FIG. 3. The needle receiving guide 304 may be positioned and oriented relative to the guide surface 306 based on the target treatment location 206. The needle receiving guide 304 may further positioned and oriented relative to the needle insertion location 202 on the external surface 104. For example, as shown in FIG. 3, the needle receiving guide 304 is positioned and oriented to align with the needle trajectory 210 extending from the needle insertion location 202 to the deeper target treatment location 206.

The patient-specific needle guide geometry 302 may also include a needle depth stop 308. The needle depth stop 308 may be positioned on the needle receiving guide 304 based on the depth of the target treatment location 206 and the needle insertion location 202. More specifically, the needle depth stop 308 may be positioned on the needle receiving guide 304 such that a needle being used to inject a deep structure in the target treatment location 206 reaches the target treatment location 206. The needle depth stop 308 may also help to prevent the needle from passing a maximum depth 310 with a margin of error of up to 1 cm. A length of the needle receiving guide 304 may also facilitate proper insertion needle of a needle. For example, the needle depth stop 308 may be on an upper end (e.g., as shown in FIG. 3) of the needle receiving guide 304. In such examples, the needle depth stop 308 may prevent a syringe associated with the needle from entering the needle receiving guide 304.

In other examples, the needle depth stop 308 may be positioned in other locations along the length of the needle receiving guide 304. In some examples, the needle depth stop 308 may be a protrusion extending from a surface of the needle receiving guide 304. The protrusion may prevent the insertion of the needle past the maximum depth 310 by preventing the syringe associated with the needle from being inserted into the needle receiving guide 304 or by only allowing the syringe to be partially inserted into the needle receiving guide 304. In other examples, the needle depth stop 308 may be associated with a diameter of the needle receiving guide 304. For example, the diameter of the needle receiving guide 304 may be smaller than a diameter of the syringe while being greater than a diameter of the needle. As a result, the needle receiving guide 304 can prevent the syringe associated with the needle from being inserted into the needle receiving guide 304. In another example, a first section of the needle receiving guide 304 may have a diameter greater than the syringe while a second section of the needle receiving guide 304 may have a diameter smaller than the syringe. In this example, the syringe may be partially inserted into the needle receiving guide 304 until stopped by the needle depth stop (e.g., the second section).

FIG. 4 is a schematic example of a patient-specific needle guide 402 according to some aspects of the present disclosure. The patient-specific needle guide 402 may be 3D printed based on the patient-specific needle guide geometry 302 shown and described above with respect to FIG. 3. Thus, the patient-specific needle guide 402 includes a 3D printed guide surface 416, a 3D printed needle depth stop 418, and a 3D printed needle receiving guide 414 that correspond to the guide surface 306, the needle depth stop 308, and the needle receiving guide 304 respectively.

In FIG. 4, the patient-specific needle guide 402 is shown on an external surface 404 of the patient. The external surface 404 corresponds to the external surface 104 of the patient that was imaged using an imaging modality and shown in 3D model 100. Due to the 3D printed guide surface 416 being created based on the guide surface 306, the 3D printed guide surface 416 is contoured to match the 3D surface geometry 204 obtained from the 3D model 100. Thus, the 3D printed guide surface 416 is contoured to match the external surface 404 of the patient. As such, the patient-specific needle guide 402 may be a negative of the external surface 404 of the patient that can match the external surface 404 of the patient when in a proper orientation and position on the external surface 404 of the patient. The proper orientation of position of the patient-specific needle guide 402 on the patient may be the position and orientation in which the needle receiving guide 414 is aligned with needle insertion location 403. This, in turn, can provide a stable surface through which a needle 412 can be inserted at a desired location and in a controlled manner.

FIG. 4 further shows the needle 412 guided in trajectory and depth into the deep anatomical structure 406 corresponding to the deep anatomical structure 106 that was imaged and shown in 3D model 100. The needle 412 can be precisely guided in trajectory and depth to the targeted deep anatomical structure 406 due to the patient-specific needle guide 402 being designed based on the information obtained from the 3D data (e.g., the target treatment location 206). The second deep anatomical structure 408 and the third deep anatomical structure 410 may correspond to the deep anatomical structures 108, 110 shown in the 3D model 100.

Due to the patient-specific needle guide 402 being constructed such that the patient-specific needle guide 402 matches the external surface 404 of the patient when in the proper position and orientation and due to the patient-specific needle guide 402 including structures that control a depth and trajectory of the needle 412 (e.g., the needle depth stop 418 and needle receiving guide 414), a safe, accurate, and efficient injection produce (e.g., injection of a nerve block or joint of target) may be performed with respect to the deep anatomical structure 406 using the patient-specific needle guide 402 with reduced or no need for intra-procedure imaging. For example, because the patient-specific needle guide 402 matches the external surface 404 of the patient when positioned and oriented in the proper position and orientation, a needle can be inserted with precision to follow a desired trajectory (e.g., a needle trajectory that avoids contacting undesired structures that would be damaged).

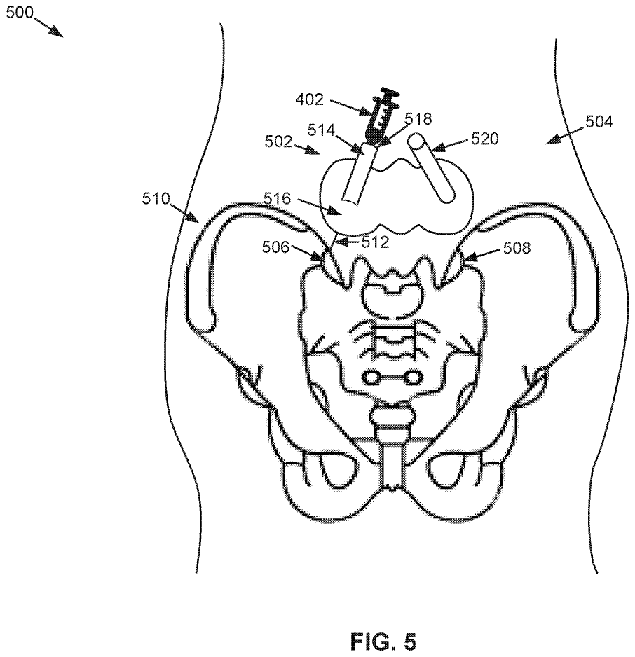

FIG. 5 is another schematic example of a patient-specific needle guide 502 according to some aspects of the present disclosure. The patient-specific needle guide 502 may be generated based on 3D data obtained from recent imaging anatomy of the patient (e.g., the pelvic region 510 of the patient) and then 3D printed. The patient-specific needle guide 502 includes a 3D printed guide surface 516, a 3D printed needle depth stop 518, and a 3D printed needle receiving guide 514.

In FIG. 5, the patient-specific needle guide 502 is shown on an entry point of the external surface 504 of the patient. The guide surface 516 may be contoured based on surface geometry identified in the 3D data and therefore may match the external surface 504 of the patient when the guide is positioned in the desired position angle and orientation. FIG. 5 further shows the needle 512 penetrating into a deep anatomical structure (e.g., sacroiliac joint 506). The needle 512 can accurately target the sacroiliac joint 506 due to the patient-specific needle guide 502 being designed based on information obtained from the 3D data (e.g., a target treatment location, external surface geometry, etc.). The patient-specific needle guide 502 also includes a second needle receiving guide 520. The second needle receiving guide 520 may be sized, positioned, and oriented to facilitate penetration of a needle in another deep anatomical structure (e.g., the contra-lateral sacroiliac joint 508).

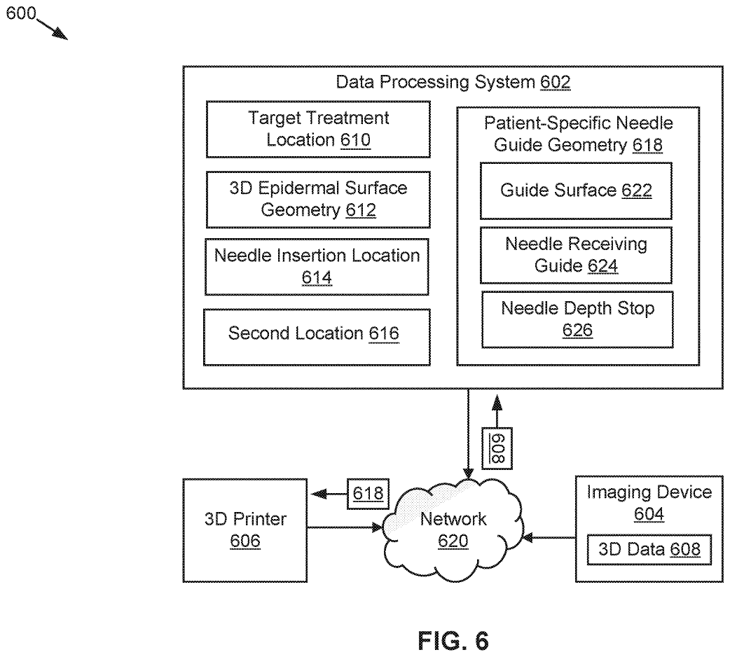

FIG. 6 is a block diagram of an example of a system 600 for preparing a patient-specific needle guide (e.g., patient-specific needle guide 402 shown in FIG. 4 or patient-specific needle guide 502 shown in FIG. 5) according to some aspects of the present disclosure. The system 600 may be a computing data such as a cloud computing environment, a distributed computing environment (e.g., an edge computing environment), or the like. The system 600 includes a data processing system 602, an imaging device 604, and a three-dimensional (3D) printer 606. The data processing system 602 may be part of the imaging device 604 or the 3D printer 606 in some examples, or the data processing system 602 may be external to and communicatively coupled with the imaging device 604 or the 3D printer 606 in other examples. For example, the data processing system 602, the 3D printer 606, and the imaging device 604 are communicatively coupled via a network 620 in FIG. 6. The network 620 may include a local area network (LAN), wide area network (WAN), the Internet, or any combination thereof.

The data processing system 602 may obtain the 3D data 608 from the imaging of a patient's anatomy via the imaging device 604. The imaging device 604 may be a CT machine and MRI machine or another suitable device. The imaging device 604 may be used to obtain a set of slices (e.g., two-dimensional cross-section images) that, individually, show two-dimensional segments of the patient's anatomy and, in combination, provide the 3D data. For example, the 3D data 608 may include measurements from the imaging device (e.g., a signal intensity or tissue density) and positioning information to indicate a location at which each measurement was obtained. In some examples, the 3D data 608 may be obtained from imaging the patient's anatomy while the patient is positioned in a treatment pose. The treatment pose may be a body positioning of the patient during a corresponding injection procedure. For example, the treatment pose of the patient may a prone position. Thus, the patient may be in the prone position during when the imaging device 604 images the patient's anatomy and thereby obtains the 3D data 608. In other examples, the treatment pose may be a different from a body positioning of the patient during imaging. For example, the patient may be in a supine position when the 3D data 608 is obtained from imaging the patient's anatomy. Then, the patient may be in a prone position during a corresponding injection procedure.

The data processing system 602 may then identify in the 3D data 608 a target treatment location 610 and a 3D surface geometry 612. To do so, the data processing system 602 may generate a 3D model of the patient's anatomy using the 3D data. Then, the data processing system 602 can identify an area of the 3D model that corresponds to a target injection site (e.g., a target anatomical structure) to identify the target treatment location 610. The data processing system 602 may further identify an area of the 3D model that corresponds to an external surface of the patient at which a needle could be inserted to reach the target injection site to identify the 3D surface geometry 612.

Moreover, the data processing system 602 may identify, in the 3D data 608, a needle insertion location 614 relative to the 3D surface geometry 612 and/or a second location 616. The needle insertion location 614 can be a subsection of the area of the 3D model corresponding to the external surface of the patient at which a needle could be inserted to reach the target injection site. The needle insertion location 614 may be identified to enable an efficient and safe injection procedure. For example, the second location 616 may be a deep anatomical structure (e.g., a blood vessel or spinal cord) to avoid during the injection to prevent bleeding, injury, or other undesirable side effects of the injection procedure. Thus, the needle insertion location 614 may be identified such that a needle trajectory (e.g., a trajectory of a needle when inserted) extending from the needle insertion location 614 to the target treatment location 610 does not pass through the second location 616.

After identifying the target treatment location 610, the 3D surface geometry 612, the needle insertion location 614, or a combination thereof, the data processing system 602 may define a patient-specific needle guide geometry 618 that includes a guide surface 622 and a needle receiving guide 624. The data processing system 602 may use the superficial target treatment location 610, the 3D surface geometry 612, the needle insertion location 614, or a combination thereof to define the guide surface 622 and the needle receiving guide 624. For example, the guide surface 622 can be a substantially thin and plate-like structure designed to rest against the surface of the patient. Therefore, the guide surface 622 may be contoured based on the 3D surface geometry 612.

Additionally, the needle receiving guide 624 may positioned and oriented relative to the guide surface 622 based on the target treatment location 610 and the needle receiving guide 624. In other words, the needle receiving guide 624 may be positioned and oriented such that, when the patient-specific needle guide geometry 618 is positioned on the surface of the patient and a needle is inserted into the needle receiving guide 624, the needle is directed through the surface in a location corresponding to the needle insertion location 614 to a structure in the target treatment location 610.

In some examples, the defined patient-specific needle guide geometry 618 further includes a needle depth stop 626 that is defined based on the target treatment location 610 and the needle insertion location 614. The needle depth stop 626 may be part of the needle receiving guide 624. The needle depth stop 626 may be designed to control insertion depth of the needle during the injection procedure. For example, the needle depth stop 626 may prevent insertion of a syringe associated with the needle into the needle receiving guide 624 to control insertion depth of the needle. In such an example, a length of the needle receiving guide 624 can be selected based on a length of the needle and a depth of the target treatment location 610.

Once the patient-specific needle guide geometry 618 is defined, the data processing system 602 may use the defined patient-specific needle guide geometry 618 to manufacture the patient-specific needle guide (e.g., patient-specific needle guide 402 shown in FIG. 4 or patient-specific needle guide 502 shown in FIG. 5). For example, the data processing system 602 may transmit the patient-specific needle guide geometry 618 to a 3D printer 606 to cause the 3D printer to 3D print the patient-specific needle guide.

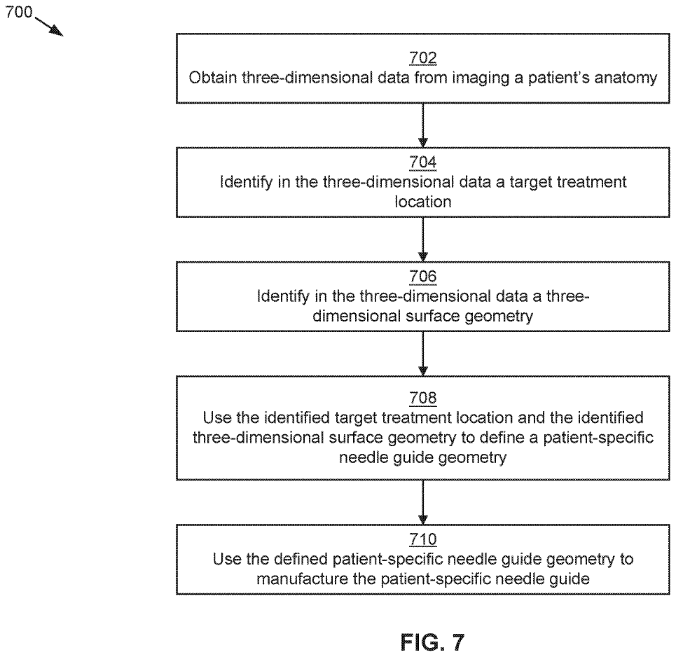

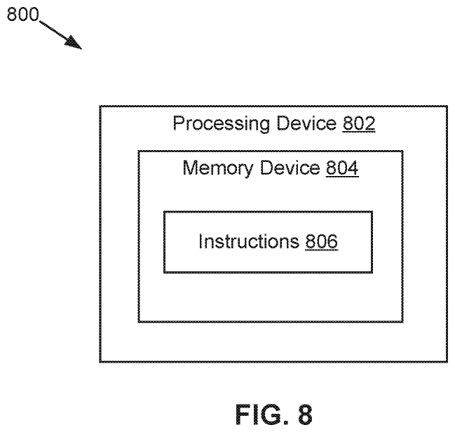

FIG. 7 is a flowchart of an example of a method 700 for preparing a patient-specific needle guide according to some aspects of the present disclosure. In one example, the processing device 802 of FIG. 8 can execute the data processing system 602 of FIG. 6 to perform one or more of the steps shown in FIG. 7. In other examples, the processing device 802 can implement more steps, fewer steps, different steps, or a different order of the steps depicted in FIG. 7. The steps of FIG. 7 are described below with reference to components discussed above in FIGS. 1-6.

At block 702, the method 700 includes obtaining three-dimensional (3D) data 608 from imaging a patient's anatomy. For example, the 3D data 608 may be obtained from images obtained via MRI or CT imaging. The images may be slices that each show a two-dimensional segment of the patient's anatomy and that are used to construct a 3D model showing the 3D data 608. An example of a 3D model 100 representative of the 3D data 608 is shown in FIGS. 1-3. The 3D data can include an imaging measurement and a position in three-dimensional space. The imaging measurement can be an intensity measurement, a density measurement, or the like. The 3D data may include an imaging measurement for each pixel of the image. The 3D may also include pixel size or other suitable metadata.

At block 704, the method 700 includes identifying in the 3D data 608 a target treatment location 610 associated with a deep anatomical structure of the patient's anatomy (e.g., deep anatomical structure 106). The target treatment location 610 can include different structures, such as a nerve or a bone joint, that is a target site for an injection procedure. The injection procedure may be a treatment in which a medication is injected into the target site. In one example, the injection procedure may involve injecting regenerative agents, steroids, lubricating medications, nerve blockers, neurolytic agents, or the like into the target site for pain management purposes.

At block 706, the method 700 includes identifying in the 3D data 608 a 3D surface geometry 612 associated with an external surface of the patient's anatomy (e.g., external surface 104). The 3D surface geometry 612 can correspond to an area of a skin surface of the patient. For example, the 3D surface geometry 612 can be a plate-like structure that is curved or otherwise shaped to rest against the area of the skin surface of the patient.

At block 708, the method 700 includes using the identified target treatment location 610 and the identified 3D surface geometry 612 to define a patient-specific needle guide geometry 618. The patient-specific needle guide geometry 618 may include at least one guide surface 622 that is contoured based on the 3D surface geometry 612. The patient-specific needle guide geometry 618 may also include a needle receiving guide 624 that is positioned and oriented relative to the at least one guide surface 622 based on the target treatment location 610.

At block 710, the methods 700 includes using the defined patient-specific needle guide geometry 618 to manufacture the patient-specific needle guide. In one example, the patient-specific needle guide geometry 618 may be transmitted to a 3D printer 606 to cause the 3D printer 606 to print the patient-specific needle guide. Once manufactured, the patient-specific needle guide may be used to guide one or more injection procedures on the patient. For example, using the patient-specific needle guide to guide an injection procedure, may include positioning the patient-specific needle guide such that the guide surface contacts the external surface of the patient and inserting a needle through the needle receiving guide such that a distal tip of the needle penetrates to the target treatment location. The needle may then be used to deliver a pharmacological agent (e.g., a nerve blocking agent or neurolytic) to the target treatment location.

The foregoing description of certain examples, including illustrated examples, has been presented only for the purpose of illustration and description and is not intended to be exhaustive or to limit the disclosure to the precise forms disclosed. Numerous modifications, adaptations, and uses thereof will be apparent to those skilled in the art without departing from the scope of the disclosure. The examples disclosed herein may be combined or rearranged to yield additional examples.

What is claimed is:

1. A method of preparing a patient-specific needle guide, the method comprising:
(a) obtaining three-dimensional data from imaging a patient's anatomy;
(b) identifying in the three-dimensional data a target treatment location associated with a deep anatomical structure of the patient's anatomy;
(c) identifying in the three-dimensional data a three-dimensional surface geometry associated with an external surface of the patient's anatomy;
(d) using the identified target treatment location and the identified three-dimensional surface geometry to define a patient-specific needle guide geometry comprising (i) at least one guide surface that is contoured based on the three-dimensional surface geometry; and (ii) a needle receiving guide that is positioned and oriented relative to the at least one guide surface based on the target treatment location; and
(e) using the defined patient-specific needle guide geometry to manufacture the patient-specific needle guide.

2. The method of claim 1, further comprising identifying in the three-dimensional data a needle insertion location relative to the three-dimensional surface geometry, and wherein the needle insertion location is also used to define the patient-specific needle guide geometry.

3. The method of claim 2, further comprising identifying in the three-dimensional data a second location associated with a second deep anatomical structure, wherein the needle insertion location is identified such that a needle trajectory extending from the needle insertion location to the target treatment location does not pass through the second location.

4. The method of claim 2, wherein the defined patient-specific needle guide geometry further comprises a needle depth stop that is defined based on the target treatment location and the needle insertion location.

5. The method of claim 1, further comprising using the manufactured patient-specific needle guide to guide an injection procedure on the patient.

6. The method of claim 5, wherein using the manufactured patient-specific needle guide to guide the injection procedure on the patient comprises positioning the patient-specific needle guide such that the guide surface contacts the external surface of the patient and inserting a needle through the needle receiving guide such that a distal tip of the needle penetrates to the deep anatomical structure associated with the target treatment location.

7. The method of claim 6, further comprising using the needle to deliver a pharmacological agent to perform a nerve block or to deliver a neurolytic to the target treatment location.

8. The method of claim 7, wherein the target treatment location comprises a nerve or a bone joint.

9. The method of claim 1, further comprising generating a three-dimensional model representative of the three-dimensional data obtained from imaging the patient's anatomy.

10. The method of claim 9, wherein the target treatment location and the three-dimensional surface geometry are identified using the three-dimensional model.

11. The method of claim 1, wherein using the defined patient-specific needle guide geometry to manufacture the patient-specific needle guide comprises using a three-dimensional (3D) printer to 3D print the patient-specific needle guide.

12. The method of claim 1, wherein obtaining the three-dimensional data from imaging the patient's anatomy comprising obtaining the three-dimensional data from a plurality of MRI images or a plurality of CT images.

13. The method of claim 12, wherein the three-dimensional data comprises an imaging measurement and a position in three-dimensional space.

14. A patient-specific needle guide comprising:
at least one guide surface that is contoured to correspond to an external surface of a patient, wherein the external surface in associated with a target treatment location in that the target treatment location is reachable by a needle from the external surface; and
a needle receiving guide that is positioned and oriented relative to the at least one guide surface based on the target treatment location.

15. The patient-specific needle guide of claim 14, wherein the at least one needle receiving guide is further positioned relative to a needle insertion location on the external surface.

16. The patient-specific needle guide of claim 15, further comprising a needle depth stop that is configured to prevent insertion of a needle past a maximum depth.

17. The patient-specific needle guide of claim 16, wherein the needle depth stop is positioned on the needle receiving guide based on the target treatment location and the needle insertion location.

18. The patient-specific needle guide of claim 15, wherein a length of the needle receiving guide is based on the target treatment location and the needle insertion location.

19. The patient-specific needle guide of claim 14, wherein a diameter of the needle receiving guide is smaller than a diameter of a syringe associated with the needle.

20. The patient-specific need guide of claim 14, further comprising a second needle receiving guide that is positioned and oriented relative to the at least one guide surface based on a second target treatment location.
